(19) United States
(12) Patent Application Publication
(10) Pub. No.: US 2024/0335204 A1
(43) Pub. Date: Oct. 10, 2024

(54) Bone Reshaping Tool and Method

(71) Applicant: Texas Scottish Rite Hospital for Children, Dallas, TX (US)

(72) Inventors: Harry K.W. Kim, Dallas, TX (US); Brad A. Niese, Southlake, TX (US); David M. Edwards, Keller, TX (US)

(21) Appl. No.: 18/628,192

(22) Filed: Apr. 5, 2024

Related U.S. Application Data
(60) Provisional application No. 63/457,872, filed on Apr. 7, 2023.

(51) Int. Cl. A61B 17/16; A61B 17/88

(52) U.S. Cl. CPC A61B 17/1668; A61B 17/8811

(57) ABSTRACT

A bone reshaping tool comprising a hollow tube comprising bone-engaging drive threads on at least a portion of an exterior surface of the hollow tube; an expandable tip at a distal end of the hollow tube, the expandable tip connected to a tip orientation knob at a proximal end of the hollow tube to rotatably orient the expandable tip; and a tip expander disposed at a distal end of the expandable tip, the tip expander disposed to be moved in and out of the expansion tip, and connected to a tip expander drawbar at the proximal end of the hollow tube.

### CROSS-REFERENCE TO RELATED APPLICATIONS

This application claims priority to U.S. Provisional Patent Application Ser. No. 63/457,872 filed on Apr. 7, 2023, the entire contents of which are hereby incorporated by reference.

### STATEMENT OF FEDERALLY FUNDED RESEARCH

None.

### TECHNICAL FIELD OF THE INVENTION

The present invention relates in general to bone reshaping. In particular, the present invention relates to bone reshaping for the treatment of Perthes Disease.

### BACKGROUND OF THE INVENTION

With any avascular necrosis of the femoral head in children and adults, including Legg Calve Perthes Disease (also known as Perthes Disease), blood flow to the femoral head is disrupted and produces osteonecrosis (bone death). When such disruption occurs, the bone weakens as it is resorbed, and physiological loads can create head deformity. This head deformity can become permanent as the healing process continues through bone remodeling. Such head deformity can lead to loss of motion, early onset osteoarthritis, and pain. There is a need for an apparatus and method for the restoration of anatomical femoral head anatomy that would result in improved range of motion, reduced onset of osteoarthritis, and reduced hip pain.

### SUMMARY OF THE INVENTION

As embodied and broadly described herein, an aspect of the present disclosure relates to a bone reshaping tool comprising, consisting essentially of, or consisting of a hollow tube comprising bone-engaging drive threads on at least a portion of an exterior surface of the hollow tube; an expandable tip at a distal end of the hollow tube, the expandable tip connected to a tip orientation knob at a proximal end of the hollow tube to rotatably orient the expandable tip; and a tip expander disposed at a distal end of the expandable tip, the tip expander disposed to be moved in and out of the expansion tip, and connected to a tip expander drawbar at the proximal end of the hollow tube. In one aspect, the expandable tip comprises a plurality of prongs, each prong fixed at a proximal end of the expandable tip and free to be moved in and out radially by the tip expander at a distal end of the expandable tip. In another aspect, the plurality of prongs is beveled to accommodate a curved anatomy of a femoral head. In another aspect, the expandable tip can be rotated without rotating the hollow tube. In another aspect, the tip expander is conical, with a conical base at a distal end of the tip expander and a conical point at a proximal end of the tip expander. In another aspect, the expandable tip is configured to elevate a subchondral bone to near anatomical geometry. In another aspect, the bone reshaping tool can be driven within a femoral head to push the subchondral bone.

As embodied and broadly described herein, another aspect of the present disclosure relates to a bone reshaping tool kit comprising, consisting essentially of, or consisting of a bone reshaping tool comprising a hollow tube comprising bone-engaging drive threads on at least a portion of an exterior surface of the hollow tube; an expandable tip at a distal end of the hollow tube, the expandable tip connected to a tip orientation knob at a proximal end of the hollow tube to rotatably orient the expandable tip; and a tip expander disposed at a distal end of the expandable tip, the tip expander disposed to be moved in and out of the expansion tip, and connected to a tip expander drawbar at the proximal end of the hollow tube; and one or more tools to manipulate the hollow tube, the expandable tip, the tip expander, or some combination. In one aspect, the expandable tip comprises a plurality of prongs, each prong fixed at a proximal end of the expandable tip and free to be moved in and out radially by the tip expander at a distal end of the expandable tip. In another aspect, the plurality of prongs is beveled to accommodate an anatomy of a femoral head. In another aspect, the expandable tip can be rotated without rotating the hollow tube. In another aspect, the tip expander is conical, with a conical base at a distal end of the tip expander and a conical point at a proximal end of the tip expander. In another aspect, the expandable tip is configured to elevate a subchondral bone to near anatomical geometry. In another aspect, the bone reshaping tool can be driven within a femoral head to push the subchondral bone.

As embodied and broadly described herein, another aspect of the present disclosure relates to a method of reshaping a femoral head comprising, consisting essentially of, or consisting of providing a patient in need of reshaping the femoral head; providing a bone reshaping tool comprising a hollow tube comprising bone-engaging drive threads on at least a portion of an exterior surface of the hollow tube; an expandable tip at a distal end of the hollow tube, the expandable tip connected to a tip orientation knob at a proximal end of the hollow tube to rotatably orient the expandable tip; and a tip expander disposed at a distal end of the expandable tip, the tip expander disposed to be moved in and out of the expansion tip, and connected to a tip expander drawbar at the proximal end of the hollow tube; introducing the bone reshaping tool into the femoral head; expanding the expandable tip to restore an anatomic shape of the femoral head; withdrawing the bone reshaping tool from the femoral head; and introducing bioresorbable bone cement or bone graft material into the femoral head to stabilize the restored femoral head.

### BRIEF DESCRIPTION OF THE DRAWINGS

For a more complete understanding of the features and advantages of the present invention, reference is now made to the detailed description of the invention along with the accompanying figures, in which:

FIG. 1A shows initial bone resorption and a mild femoral head flattening in a case of Perthes Disease. FIG. 1B shows advanced bone resorption and collapse of the femoral head. FIG. 1C shows permanent deformity and osteoarthritis.

FIG. 2A shows an example of a normal pig cadaver femoral head. FIG. 2B shows the femoral head seen in FIG. 2A after inducing femoral head collapse to test the present invention.

FIG. 3A shows an embodiment of a bone reshaping tool with an expandable tip according to the present invention. FIG. 3B shows a tip of the bone reshaping tool in an unexpended state.

FIG. 3C shows the tip of the bone reshaping tool in a partially expanded state. FIG. 3D shows the tip of the bone reshaping tool in a fully expanded state.

FIG. 4A shows an embodiment of the bone reshaping tool of the present invention introduced into a collapsed femoral head, like the collapsed head shown in FIG. 2B. FIG. 4B shows the expandable tip of the bone reshaping tool in the fully expanded state and driven proximally to restore the shape of the femoral head. FIG. 4C shows the femoral head largely retaining its restored shape after the tip is unexpanded and withdrawn. FIG. 4D shows the introduction of resorbable bone cement to stabilize the restored femoral head.

FIG. 5A shows another embodiment of a bone reshaping tool according to the present invention. FIG. 5B shows the bone reshaping tool introduced into a diseased femoral head.

FIGS. 6A-6E show various views of another embodiment of the present invention, bone reshaping tool 600.

FIGS. 7A-7G show images related to a use of an embodiment of the present invention, the bone reshaping tool, to treat femoral head osteonecrosis and collapse in a 12-year-old female patient, due to sickle-cell disease.

FIGS. 8A-8F show images related to a use of an embodiment of the present invention, the bone reshaping tool, to treat femoral head osteonecrosis and collapse in a 16-year-old female patient.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

Restoration of the anatomic shape of a femoral head in a case of avascular necrosis such as Perthes Disease can improve range of motion and reduce the contact stresses that can lead to early onset osteoarthritis. Using bioresorbable bone cement, bone allograft, bone autograft, or synthetic bone graft substitutes such as calcium-based, magnesium-based, or hydroxyapatite-based synthetic bone graft substitutes (referred to herein generally as “bioresorbable bone cement or bone graft material”) permits the revascularization and bone reformation that will lead to long term hip health. Femoral head restoration and stabilization will lead to shorter recovery times and a more aggressive return to a play regimen (for children) and the associated health benefits of increased physical activity. These advantages can be achieved with the present invention with minimally invasive surgery and minimal bone loss during surgery.

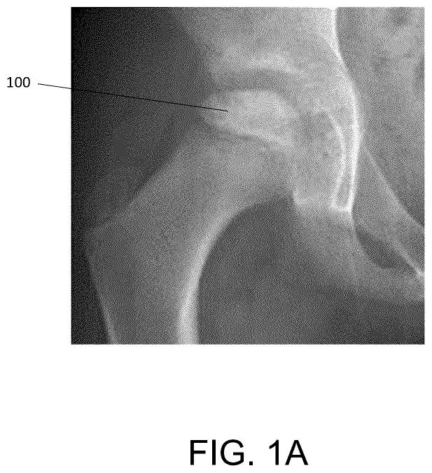

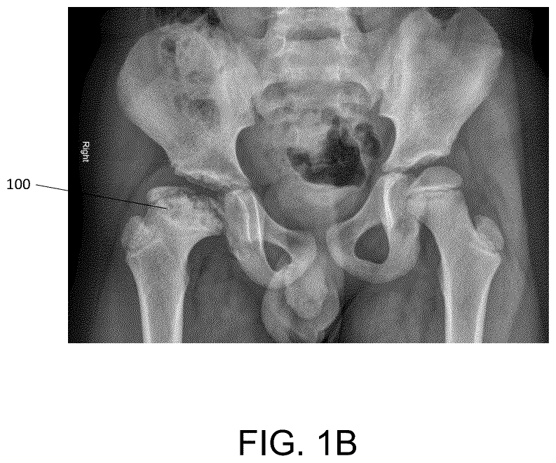

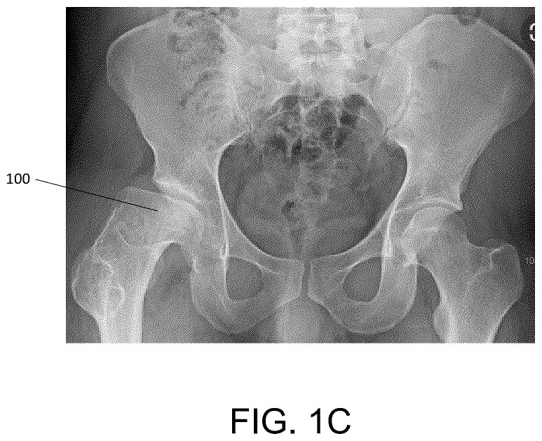

FIG. 1A shows initial bone resorption and mild femoral head flattening in a femoral head 100 in a case of Perthes Disease. FIG. 1B shows advanced bone resorption and collapse and deformation of the femoral head 100. FIG. 1C shows permanent deformity and osteoarthritis of the femoral head 100.

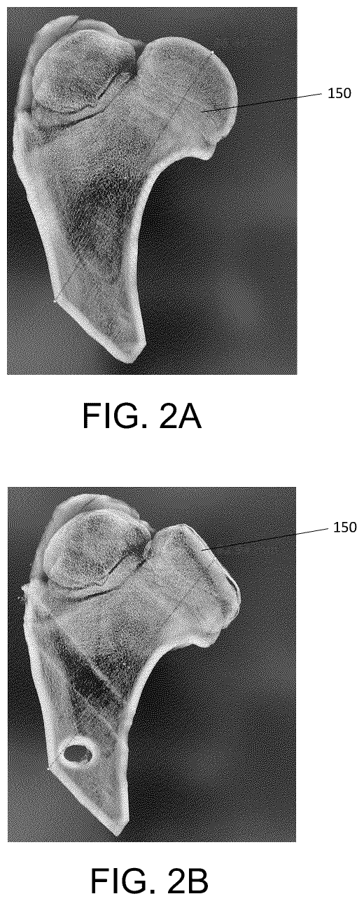

FIG. 2A shows an example of a normal pig cadaver femoral head 150. FIG. 2B shows the femoral head 150 seen in FIG. 2A after inducing femoral head collapse to test the present invention.

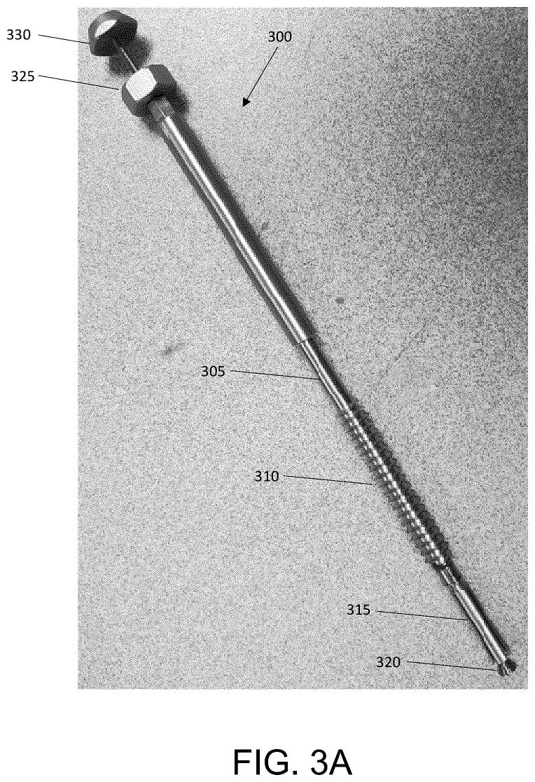

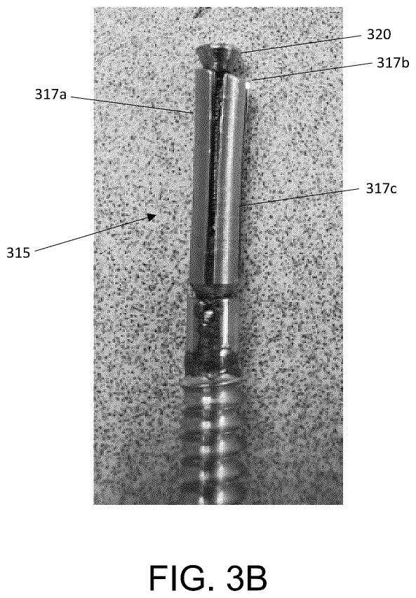

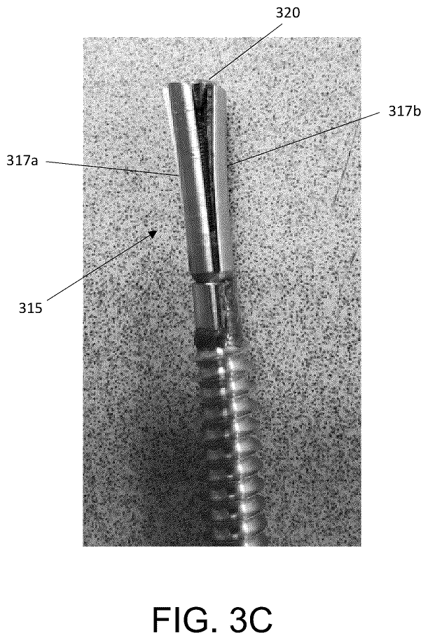

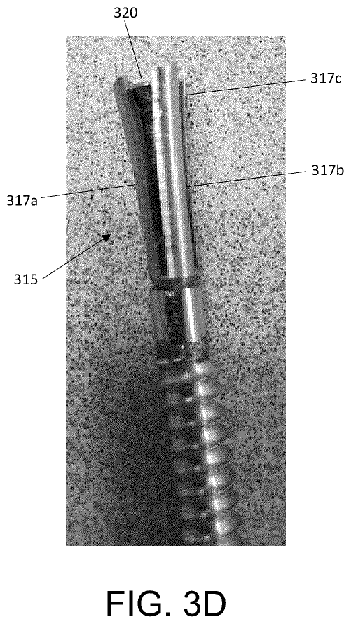

FIG. 3A shows an embodiment of a bone reshaping tool 300 with an expandable tip according to the present invention. The bone reshaping tool 300 includes a hollow tube 305 and bone-engaging drive threads 310 on at least a portion of the exterior surface of the hollow tube 305. The bone reshaping tool 300 further includes an expandable tip 315 disposed at a distal end and a tip expander 320 disposed at the distal end, disposed to be drawn into and moved out of the expandable tip 315. In addition, the bone reshaping tool 300 includes a tip orientation knob 325 at a proximal end and connected to the expandable tip 315 and a tip expander drawbar 330 at the proximal end and connected to the tip expander 320. The tip orientation knob 325 is disposed and connected the expandable tip 315 to allow an operator of the bone reshaping tool 300 to rotate the expandable tip 315 without rotating the hollow tube 305. The tip expander drawbar 330 is disposed and connected to the tip expander 320 to allow an operator of the bone reshaping tool 300 to move the tip expander 320 in and out of the expandable tip 315 longitudinally to move the distal ends of the prongs 317 (See FIG. 3B) in and out radially. Expansion of the expandable tip 315 can elevate a subchondral bone to near-anatomical geometry, and the bone reshaping tool can be driven within the femoral head to push the subchondral bone. FIG. 3B shows the expansion tip 315 of the bone reshaping tool 300 in an unexpanded state, with the tip expander 320 completely outside the expandable tip 315. The expandable tip 315 includes a plurality of prongs 317 (three exemplary prongs 317a-c are shown) such that the prongs are fixed at a proximate end of the expandable tip 315 and free at a distal end of the expandable tip 315 to be moved radially in and out by the tip expander 320. The prongs 317 can be beveled or angled at the distal end of the expandable tip 315 to accommodate the anatomy of the femoral head. FIG. 3C shows the expandable tip 315 of the bone reshaping tool 300 in a partially expanded state, with the tip expander 320 retracted within the expandable tip 315. Prongs 317a, b are shown; prong 317c is not visible and is not shown. FIG. 3D shows the expandable tip 315 of the bone reshaping tool 300 in a fully expanded state, with the tip expander 320 retracted further within the expandable tip 315.

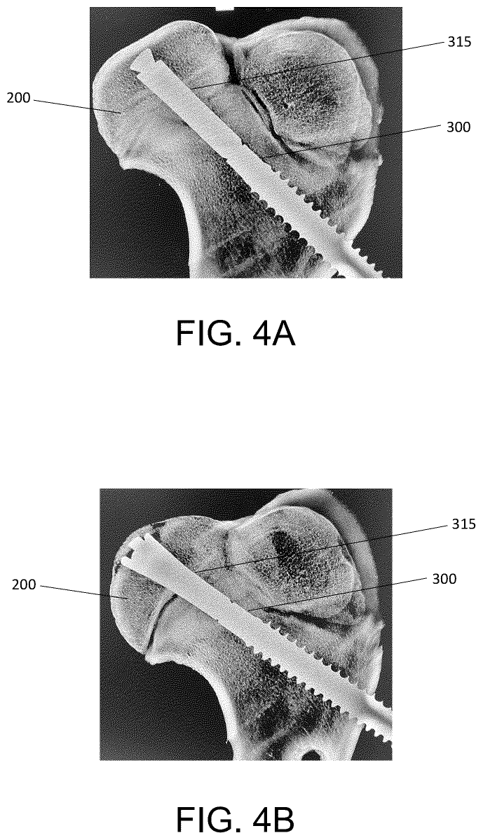

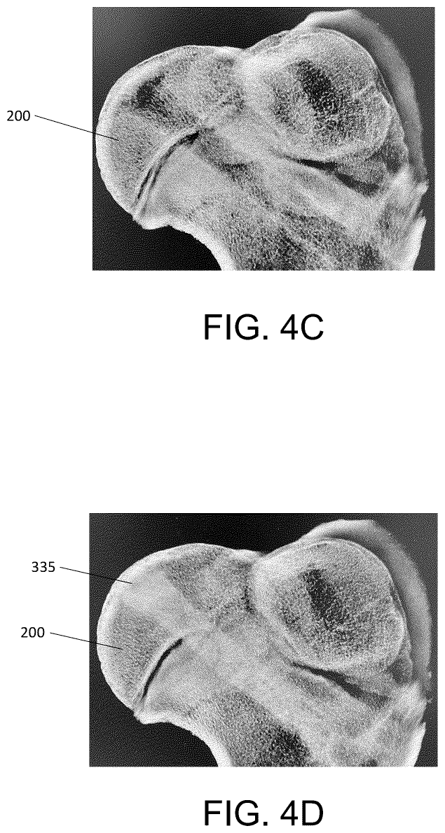

FIG. 4A shows an embodiment of the bone reshaping tool 300 of the present invention introduced into a collapsed, deformed femoral head 200, with the expandable tip 315 in an unexpanded state. FIG. 4B shows the expandable tip 315 of the bone reshaping tool 300 in the fully expanded state and driven proximally to restore the shape of the femoral head 200. FIG. 4C shows the femoral head 200 largely retaining its restored shape after the expandable tip 315 (not shown) is unexpanded and withdrawn. FIG. 4D shows the introduction of a bioresorbable bone cement or bone graft material 335 to stabilize the restored femoral head 200.

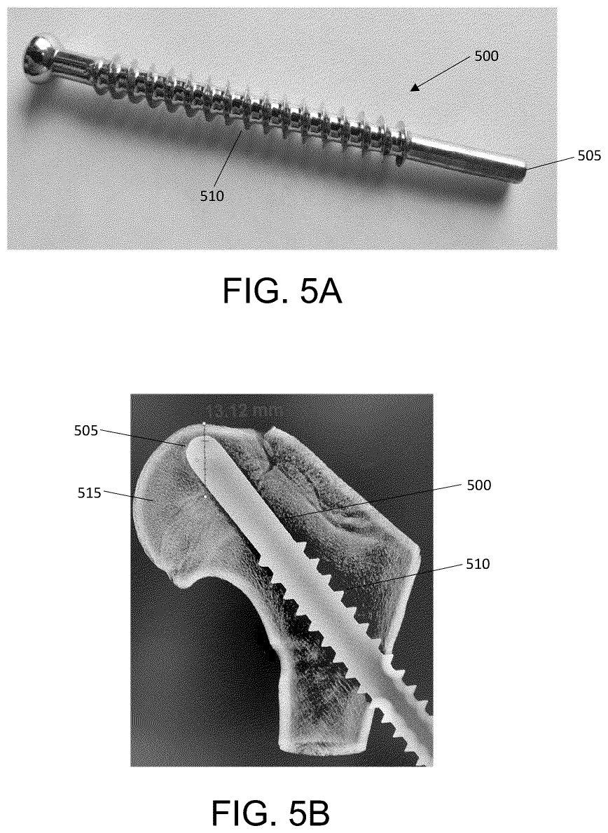

FIG. 5A shows another embodiment of a bone reshaping tool 500 according to the present invention. The bone reshaping tool has a blunt tip 505 and bone-engaging drive threads 510. FIG. 5B shows the bone reshaping tool 500 introduced into a collapsed, deformed femoral head 515, showing the blunt tip 505 and the bone-engaging drive threads 510. The bone reshaping tool 500 can be withdrawn from the femoral head 515 and bioresorbable bone cement or bone graft material (not shown) can be introduced to stabilize the restored femoral head 515.

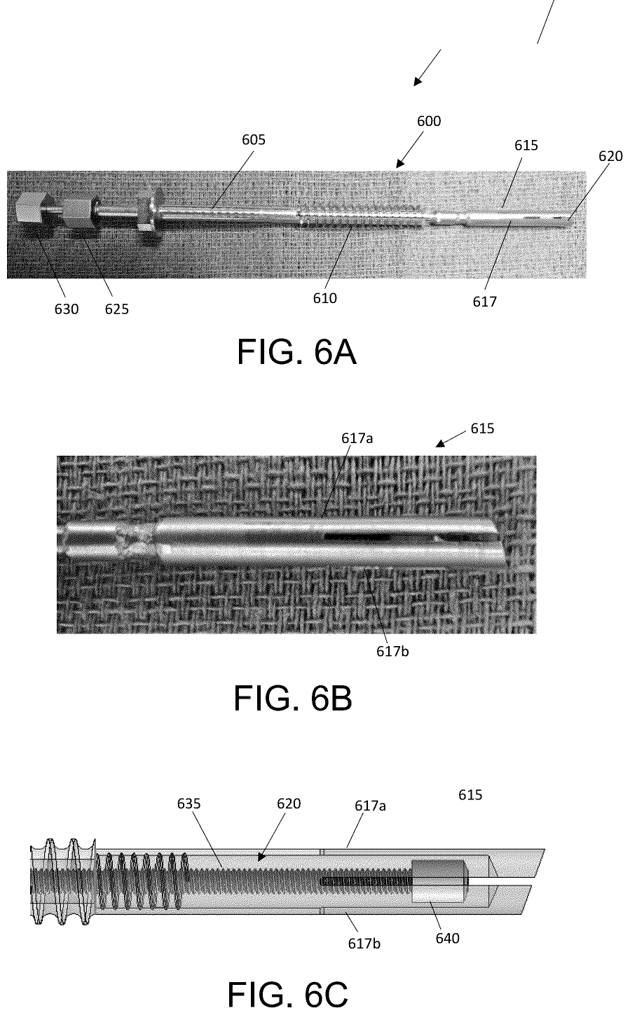

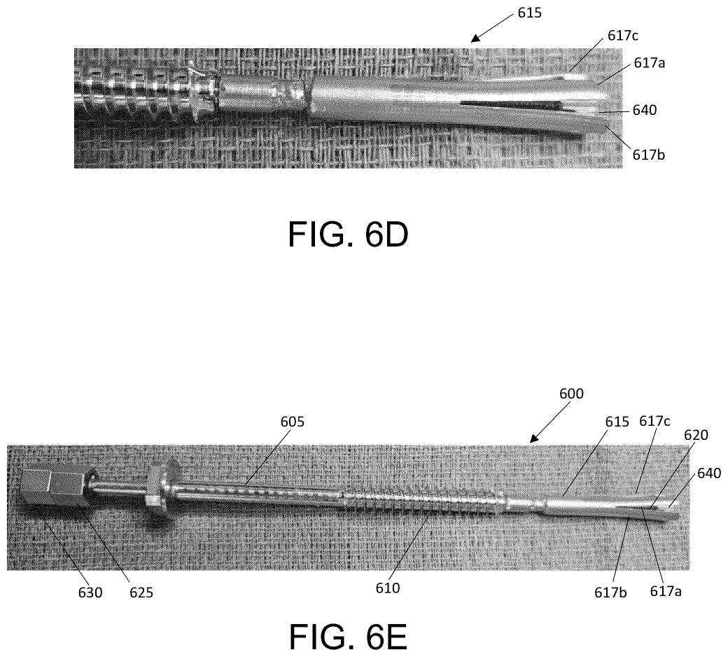

FIGS. 6A-6E show various views of another embodiment of the present invention, bone reshaping tool 600. The bone reshaping tool 600 includes a hollow tube 605 and bone-engaging drive threads 610 on at least a portion of the exterior surface of the hollow tube 605. The bone reshaping tool 600 further includes an expandable tip 615 disposed at a distal end and a tip expander 620 disposed at the distal end, disposed to be drawn into and moved out of the expansion tip 615. In addition, the bone reshaping tool 600 includes a tip orientation knob 625 at a proximal end and connected to the expandable tip 615 and a tip expander drawbar 630 at the proximal end and connected to the tip expander 620. The tip orientation knob 625 is disposed and connected the expandable tip 615 to allow an operator of the bone reshaping tool 600 to rotate the expandable tip 615 without rotating the hollow tube 605. The tip expander drawbar 630 is disposed and connected to the tip expander 620 to allow an operator of the bone reshaping tool 600 to move the tip expander 620 in and out of the expandable tip 615 longitudinally to move the distal ends of the prongs 617 in and out radially. Expansion of the expandable tip 615 can elevate a subchondral bone to near-anatomical geometry, and the bone reshaping tool can be driven within the femoral head to push the condyle. FIG. 6B shows the expansion tip 615 of the bone reshaping tool 600 in an unexpanded state, with the tip expander 620 (not shown) completely inside the expandable tip 615. The expandable tip 615 includes a plurality of prongs 617 (two exemplary prongs 617a, 617b are shown) such that the prongs are fixed at a proximate end of the expandable tip 615 and free at a distal end of the expandable tip 615 to be moved radially in and out by the tip expander 620 (not shown). The prongs 617 are shown beveled or angled at the distal end of the expandable tip 315 to accommodate the anatomy of the femoral head. FIG. 6C shows an interior of the expandable tip 615 of the bone reshaping tool 600 in an unexpanded state, with the tip expander 620 including a threaded drive rod 635 and an expander head 640 disposed inside the prongs 167a, 617b. FIG. 6D shows the expandable tip 615 in expanded state, with the expander head 640 drawn into the expandable tip 615 to move the prongs 617a-c radially outward at their distal ends (in FIG. 6D, three exemplary prongs 617a-c are shown). FIG. 6E shows another view of the bone reshaping tool 600, with the hollow tube 605, the bone-engaging drive threads 610, the expandable tip 615 in an expanded state with the prongs 617a-c moved radially outward at their distal ends, the tip expander 620 with expander head 640, the tip orientation knob 625, and the tip expander drawbar 635. The bone reshaping tool 600 may also include a positive stop (not shown) at the proximate end to prevent an over-deploy of the prongs 617a-c.

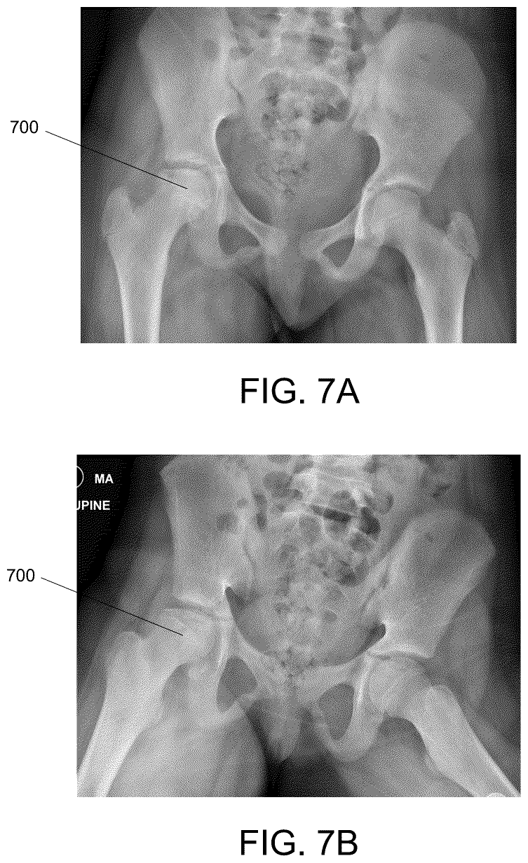

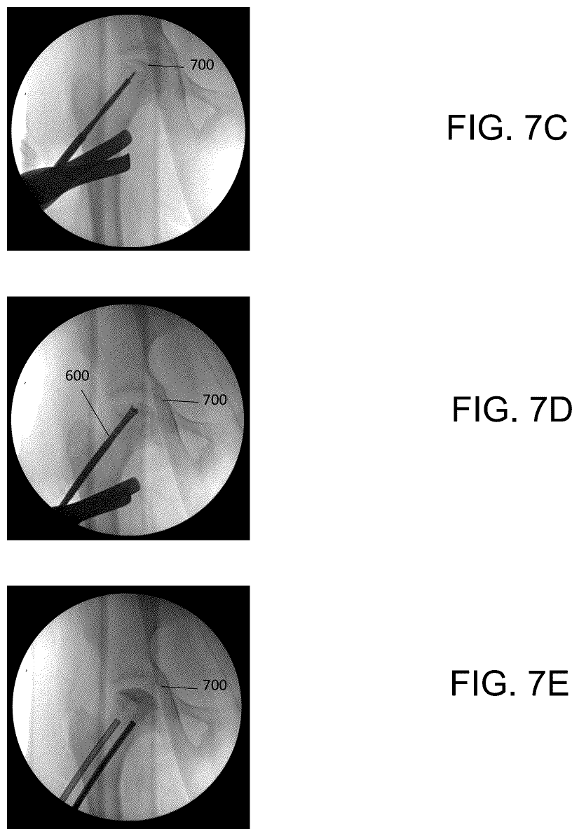

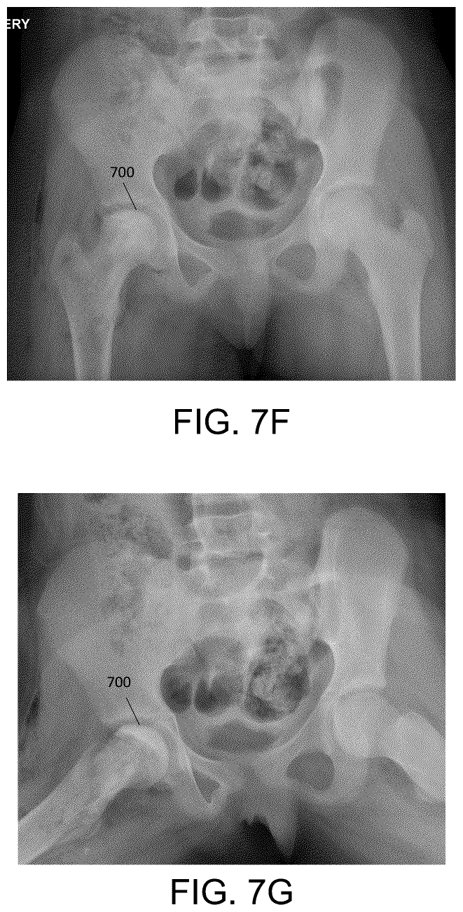

FIGS. 7A-7G show images related to a use of an embodiment of the present invention, the bone reshaping tool 600, to treat femoral head osteonecrosis and collapse in a 12-year-old female patient, due to sickle-cell disease. FIGS. 7A and 7B each show an image of a collapsed femoral head 700 before treatment. FIGS. 7C-7E show fluoroscopic images of the femoral head 700 made during a surgery with the bone reshaping tool 600, with FIG. 7C showing the insertion of a guide wire and drilling with a 5 mm cannulated drill, FIG. 7D showing a use of the bone reshaping tool 600 to raise the femoral head 700, and FIG. 7E showing back-filling with bioresorbable bone cement or bone graft material to stabilize the restored femoral head 700. FIGS. 7F and 7G each show an image of the femoral head 700 with an improved shape.

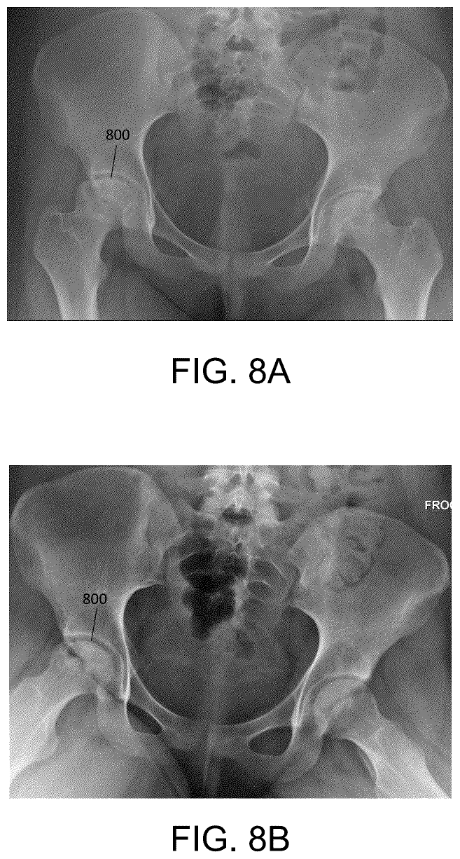

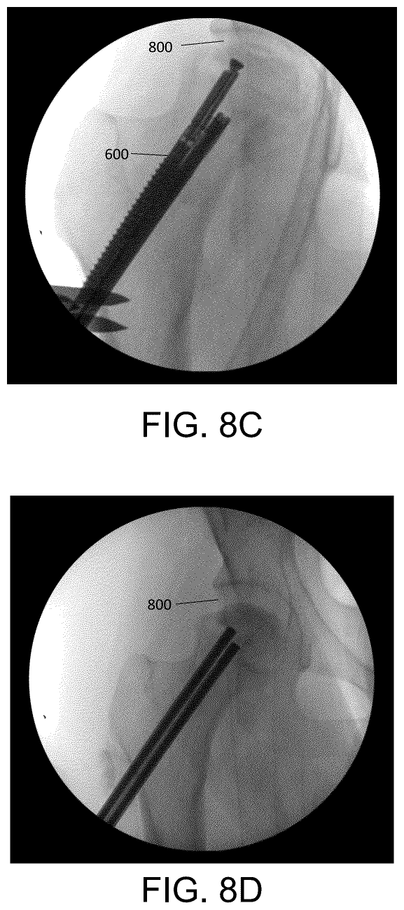

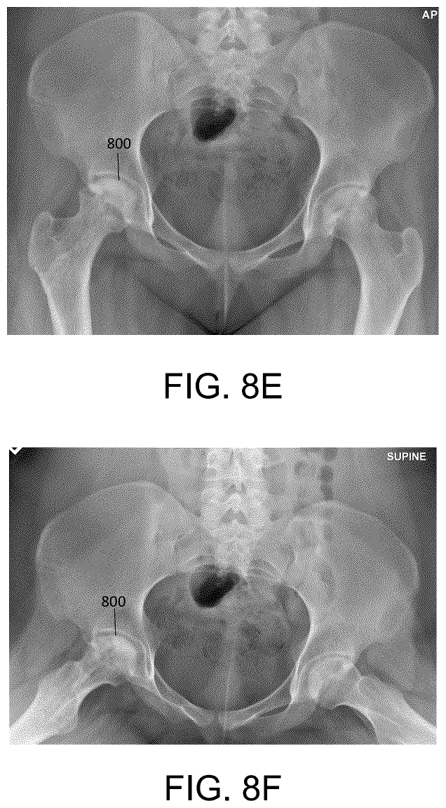

FIGS. 8A-8F show images related to a use of an embodiment of the present invention, the bone reshaping tool 600, to treat femoral head osteonecrosis and collapse in a 16-year-old female patient. FIGS. 8A and 8B each show an image of a collapsed femoral head 800 before treatment. FIGS. 8C-8D show fluoroscopic images made during a surgery with the bone reshaping tool 600, with FIG. 8C showing a use of the bone reshaping tool 600 to raise the femoral head 800, and FIG. 8D showing back-filling with bioresorbable bone cement or bone graft material to stabilize the restored femoral head 800. FIGS. 8E and 8F each show an image of the femoral head 800 with an improved shape.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112 (f) as it exists on the date of filing hereof unless the words “means for” or “step for” are explicitly used in the particular claim.

What is claimed is:

1. A bone reshaping tool comprising:
a hollow tube comprising bone-engaging drive threads on at least a portion of an exterior surface of the hollow tube;
an expandable tip at a distal end of the hollow tube, the expandable tip connected to a tip orientation knob at a proximal end of the hollow tube to rotatably orient the expandable tip; and
a tip expander disposed at a distal end of the expandable tip, the tip expander disposed to be moved in and out of the expansion tip, and connected to a tip expander drawbar at the proximal end of the hollow tube.

2. The bone reshaping tool of claim 1, wherein the expandable tip comprises a plurality of prongs, each prong fixed at a proximal end of the expandable tip and free to be moved in and out radially by the tip expander at a distal end of the expandable tip.

3. The bone reshaping tool of claim 2, wherein the plurality of prongs is beveled to accommodate an anatomy of a femoral head.

4. The bone reshaping tool of claim 1, wherein the expandable tip can be rotated without rotating the hollow tube.

5. The bone reshaping tool of claim 1, wherein the tip expander is conical, with a conical base at a distal end of the tip expander and a conical point at a proximal end of the tip expander.

6. The bone reshaping tool of claim 1, wherein the expandable tip is configured to elevate a subchondylar bone to near anatomical geometry.

7. The bone reshaping tool of claim 1, wherein the bone reshaping tool can be driven within a femoral head to push a condyle.

8. A bone reshaping tool kit comprising:
a bone reshaping tool comprising:
a hollow tube comprising bone-engaging drive threads on at least a portion of an exterior surface of the hollow tube;
an expandable tip at a distal end of the hollow tube, the expandable tip connected to a tip orientation knob at a proximal end of the hollow tube to rotatably orient the expandable tip; and
a tip expander disposed at a distal end of the expandable tip, the tip expander disposed to be moved in and out of the expansion tip, and connected to a tip expander drawbar at the proximal end of the hollow tube; and
one or more tools to manipulate the hollow tube, the expandable tip, the tip expander, or some combination.

9. The bone reshaping tool kit of claim 8, wherein the expandable tip comprises a plurality of prongs, each prong fixed at a proximal end of the expandable tip and free to be moved in and out radially by the tip expander at a distal end of the expandable tip.

10. The bone reshaping tool kit of claim 9, wherein the plurality of prongs is beveled to accommodate an anatomy of a femoral head.

11. The bone reshaping tool kit of claim 8, wherein the expandable tip can be rotated without rotating the hollow tube.

12. The bone reshaping tool kit of claim 8, wherein the tip expander is conical, with a conical base at a distal end of the tip expander and a conical point at a proximal end of the tip expander.

13. The bone reshaping tool kit of claim 8, wherein the expandable tip is configured to elevate a subchondylar bone to near anatomical geometry.

14. The bone reshaping tool kit of claim 8, wherein the bone reshaping tool can be driven within a femoral head to push a condyle.

15. A method of reshaping a femoral head comprising:
providing a patient in need of reshaping the femoral head;
providing a bone reshaping tool comprising:
a hollow tube comprising bone-engaging drive threads on at least a portion of an exterior surface of the hollow tube;
an expandable tip at a distal end of the hollow tube, the expandable tip connected to a tip orientation knob at a proximal end of the hollow tube to rotatably orient the expandable tip; and
a tip expander disposed at a distal end of the expandable tip, the tip expander disposed to be moved in and out of the expansion tip, and connected to a tip expander drawbar at the proximal end of the hollow tube;
introducing the bone reshaping tool into the femoral head;
expanding the expandable tip to restore an anatomic shape of the femoral head;
withdrawing the bone reshaping tool from the femoral head; and
introducing bioresorbable bone cement or bone graft material into the femoral head to stabilize the restored femoral head.

16. The method of claim 15, wherein the expandable tip comprises a plurality of prongs, each prong fixed at a proximal end of the expandable tip and free to be moved in and out radially by the tip expander at a distal end of the expandable tip.

17. The method of claim 16, wherein the plurality of prongs is beveled to accommodate an anatomy of a femoral head.

18. The method of claim 15, wherein the expandable tip can be rotated without rotating the hollow tube.

19. The method of claim 15, wherein the tip expander is conical, with a conical base at a distal end of the tip expander and a conical point at a proximal end of the tip expander.

20. The method of claim 15, wherein the expandable tip is configured to elevate a subchondylar bone to near anatomical geometry.

21. The method of claim 15, wherein the bone reshaping tool can be driven within a femoral head to push a condyle.
